(19) United States
(12) Patent Application Publication
(10) Pub. No.: US 2026/0091187 A1
(43) Pub. Date: Apr. 2, 2026

(54) FLUID-DRIVEN MECHANICAL VENTILATOR WITH ACCUMULATOR

(71) Applicant: IPLEXXUS HOLDING, LLC, Freeland, MI (US)

(72) Inventor: David A. DZIOBA, Freeland, MI (US)

(21) Appl. No.: 19/342,550

(22) Filed: Sep. 27, 2025

Related U.S. Application Data
(60) Provisional application No. 63/701,286, filed on Sep. 30, 2024.
(60) Provisional application No. 63/811,011, filed on May 23, 2025.

(51) Int. Cl. A61M 16/00; A61M 16/20

(52) U.S. Cl. CPC A61M 16/0075; A61M 16/0003; A61M 16/208

(57) ABSTRACT

A mechanical ventilator includes an oscillating flow controller, a respiratory interface, and an accumulator having a first variable volume chamber fluidly coupled to a bi-directional fluid port of the flow control valve, a second variable volume chamber fluidly coupled to the respiratory interface, a displaceable diaphragm fluidly separating the first variable volume chamber from the second variable volume chamber, and a biasing element configured to bias the diaphragm in the direction of the first variable volume chamber. In use, the oscillating flow controller provides an oscillating fluid output to the accumulator, thereby causing the accumulator to oscillate between first and second states, wherein the accumulator draws breathing air into the second variable volume chamber when transitioning from the second state to the first state, and wherein the accumulator forces breathing air from the second variable volume chamber to the respiratory interface when transitioning from the first state to the second state.

### CROSS-REFERENCE TO RELATED APPLICATIONS

This application claims benefit under 35 U.S.C. § 119 of U.S. Provisional Patent Application No. 63/701,286 , filed Sep. 30, 2024, and U.S. Provisional Patent Application No. 63/811,011, filed May 23, 2025, and incorporates by reference the disclosures thereof in their entireties. This application also incorporates by reference in its entirety the disclosure of U.S. patent application Ser. No. 19/081,559, filed Mar. 17, 2025.

### BACKGROUND OF THE DISCLOSURE

Mechanical ventilators are known in the art. A mechanical ventilator may be used to help a person breathe when the person is not able to do so on his or her own. Conventional mechanical ventilators typically are complex, expensive, heavy, and bulky. Given the expense in purchasing, maintaining, and storing mechanical ventilators, situations may arise where the need for them outstrips the supply thereof. This phenomenon became particularly evident during the early stages of the COVID-19 pandemic.

Also, conventional mechanical ventilators typically require electrical power to operate. As such, they typically are not sufficiently portable to be used outside a hospital setting. Thus, they typically are not available for use by emergency medical technicians (EMTs) or military medics in the field. Further, their complexity could hinder field use, even if electrical power were available to operate them there.

It would be desirable to provide a relatively low-cost mechanical ventilator that is portable, simple to use, and operable without electrical power.

### SUMMARY OF THE DISCLOSURE

In embodiments, a mechanical ventilator may include an oscillating flow controller, an accumulator, and a respiratory interface.

The oscillating flow controller may include a supply port, a first bi-directional port, and an exhaust port, and the oscillating flow controller may be configured to receive pressurized fluid through the supply port, selectively output the pressurized fluid through the first bi-directional port, selectively receive the pressurized fluid through the first-bi-directional port, and selectively output the pressurized fluid though the exhaust port.

The accumulator may include a housing, a first variable volume chamber defined within the housing, a second variable volume chamber defined within the housing, a diaphragm separating the first variable volume chamber from the second variable volume chamber, a biasing element configured to bias the diaphragm toward the first variable volume chamber, a bi-directional fluid port configured to admit fluid to the first variable volume chamber from the first bi-directional port of the control valve, and to relieve fluid from the first variable volume chamber to the first bi-directional port of the control valve, a fluid inlet port configured to admit breathing air to the second variable volume chamber, and a fluid outlet port configured to relieve breathing air from the second variable volume chamber.

The respiratory interface may include a breathing air conduit. A first end of the breathing air conduit may be fluidly coupled to the fluid outlet port, and a second end of the breathing air conduit may be configured to supply the breathing air from the fluid outlet port to a respiratory system of a user.

The oscillating flow controller may be configured to alternate between a first state in which the oscillating flow controller enables flow of the pressurized fluid from the supply port through the first bi-directional port to the first variable volume chamber and disables flow of the pressurized breathing air from the supply port and the first bi-directional port to the exhaust port, and a second state in which the oscillating flow controller enables flow of the pressurized fluid from the first variable volume chamber through the first bi-directional port to the exhaust port and disables flow of the pressurized breathing air from the supply port to the first bi-directional port and the exhaust port. The oscillating flow controller also may be configured to cyclically change state between the first state and the second state in response to the pressurized fluid flowing therethrough.

### BRIEF DESCRIPTION OF THE DRAWINGS

FIG. 1 is a side cross-sectional view of an illustrative oscillating flow controller having first and second armatures, first and second pilot chambers, and first and second movable pressure barriers, with the first and second armatures in respective first positions according to the present disclosure;

FIG. 2 is a side cross-sectional view of the oscillating flow controller of FIG. 1 with the first and second armatures in respective second positions;

FIG. 3A is a perspective view of the first and second armatures of the oscillating flow controller of FIG. 1;

FIG. 3B is a perspective view of the first and second armatures of the oscillating flow controller of FIG. 1 further showing first and second travel limiters operably associated with the second armature;

FIG. 3C is a perspective view of the first and second armatures and first and second travel limiters of the oscillating flow controller of FIG. 1 wherein the first and second movable pressure barriers are embodied as first and second flexible diaphragms connected to the first and second travel limiters, respectively;

FIG. 3D is a perspective view of the first and second movable pressure barriers and the first and second travel limiters of the oscillating flow controller of FIG. 1 apart from the first and second armatures thereof;

FIG. 4A is a side elevation view of the first and second armatures, the first and second movable pressure barriers, and the first and second travel limiters of the oscillating flow controller of FIG. 1, wherein the first and second movable pressure barriers are embodied as first and second flexible diaphragms;

FIG. 4B is a side elevation view of first and second armatures of the oscillating flow controller of FIG. 1, wherein the first and second movable pressure barriers are embodied as first and second flexible bellows;

FIG. 5A is a perspective view of the first armature of the oscillating flow controller of FIG. 1;

FIG. 5B is an exploded perspective view of the first armature of the oscillating flow controller of FIG. 1;

FIG. 6A is a perspective view of the second armature of the oscillating flow controller of FIG. 1;

FIG. 6B is an exploded perspective view of the second armature of the oscillating flow controller of FIG. 1;

FIG. 7 is a perspective view of a first permanent magnet of the first armature concentrically surrounding a second permanent magnet of the second armature of the oscillating flow controller of FIG. 1 wherein respective magnetic centers of the first and second permanent magnets are aligned;

FIG. 8A is a block diagram showing schematically the oscillating flow controller of FIG. 1 in combination with a source of pressurized fluid connected to a fluid supply port thereof, a first external accumulator connected to a first bi-directional fluid port thereof, and a second external accumulator connected to a second bi-directional fluid port thereof;

FIG. 8B is a block diagram similar to the block diagram of FIG. 8A, further showing a fluid outlet port of the oscillating flow controller fluidly coupled to a fluid inlet of the source of pressurized fluid and to a vacuum breaker;

FIG. 9 is a side cross-sectional view of the oscillating flow controller of FIG. 1 in combination with the source of pressurized fluid connected to the fluid supply port thereof, a first external accumulator connected to the first bi-directional fluid port thereof, and a second external accumulator connected to the second bi-directional fluid port thereof, with the first and second armatures of the oscillating flow controller in respect first positions;

FIG. 10 is a side cross-sectional view of the oscillating flow controller of FIG. 1 in combination with the source of pressurized fluid connected to the fluid supply port thereof, the first external accumulator connected to the first bi-directional fluid port thereof, and the second external accumulator connected to the second bi-directional fluid port thereof, with the first and second armatures of the oscillating flow controller in respect second positions;

FIGS. 11-18 are side cross-sectional views of the oscillating flow controller of FIG. 1 in an illustrative sequence of operational states when used in an illustrative manner in combination with the source of pressurized fluid and the first and second external accumulators, wherein:

FIG. 11 shows the oscillating flow controller of FIG. 1 in a first operational state;

FIG. 12 shows the oscillating flow controller of FIG. 1 in a second operational state;

FIG. 13 shows the oscillating flow controller of FIG. 1 in a third operational state;

FIG. 14 shows the oscillating flow controller of FIG. 1 in a fourth operational state;

FIG. 15 shows the oscillating flow controller of FIG. 1 in a fifth operational state;

FIG. 16 shows the oscillating flow controller of FIG. 1 in a sixth operational state;

FIG. 17 shows the oscillating flow controller of FIG. 1 in a seventh operational state;

FIG. 18 shows the oscillating flow controller of FIG. 1 in an eighth operational state;

FIGS. 19A-19D are timing diagrams reflecting illustrative pressurization and depressurization of one or more external accumulators fluidly connected to the oscillating flow controller of FIG. 1;

FIG. 20 shows schematically a mechanical ventilator including the oscillating flow controller of FIG. 1 in combination with a bellows-type accumulator fluidly connected to the first bi-directional port of the oscillating flow controller and a first respiratory interface fluidly connected to a fluid output of the accumulator via an intervening directional flow control valve, wherein the second bi-directional port of the oscillating flow controller is blocked, and wherein a source of pressurized fluid is fluidly connected to the fluid supply port of the flow control valve;

FIG. 21A shows the accumulator of FIG. 20 in detail in a first operational state;

FIG. 21B shows the accumulator of FIG. 20 in detail in a second operational state;

FIG. 22A shows schematically the directional flow control valve of FIG. 20 in a first operational state; and

FIG. 22B shows schematically the directional flow control valve of FIG. 20 in a second operational state.

### DETAILED DESCRIPTION OF THE DRAWINGS

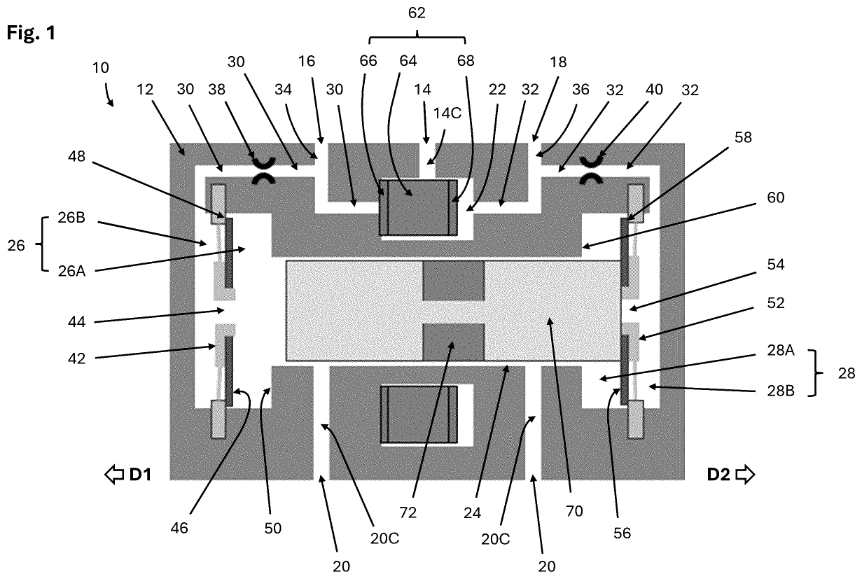

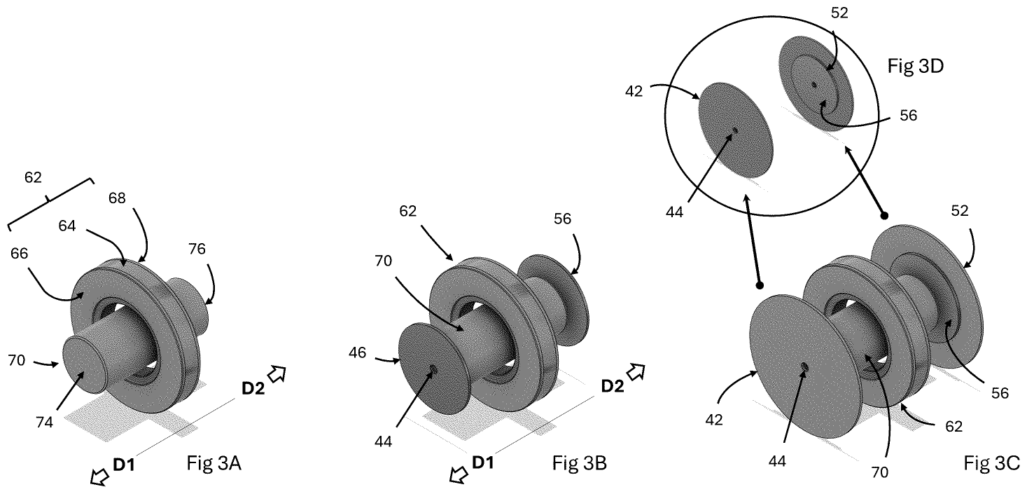

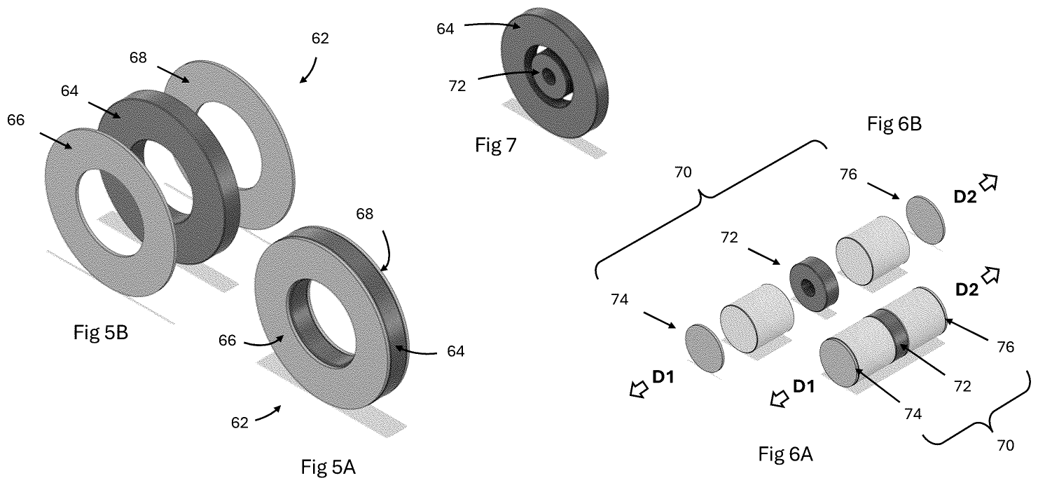

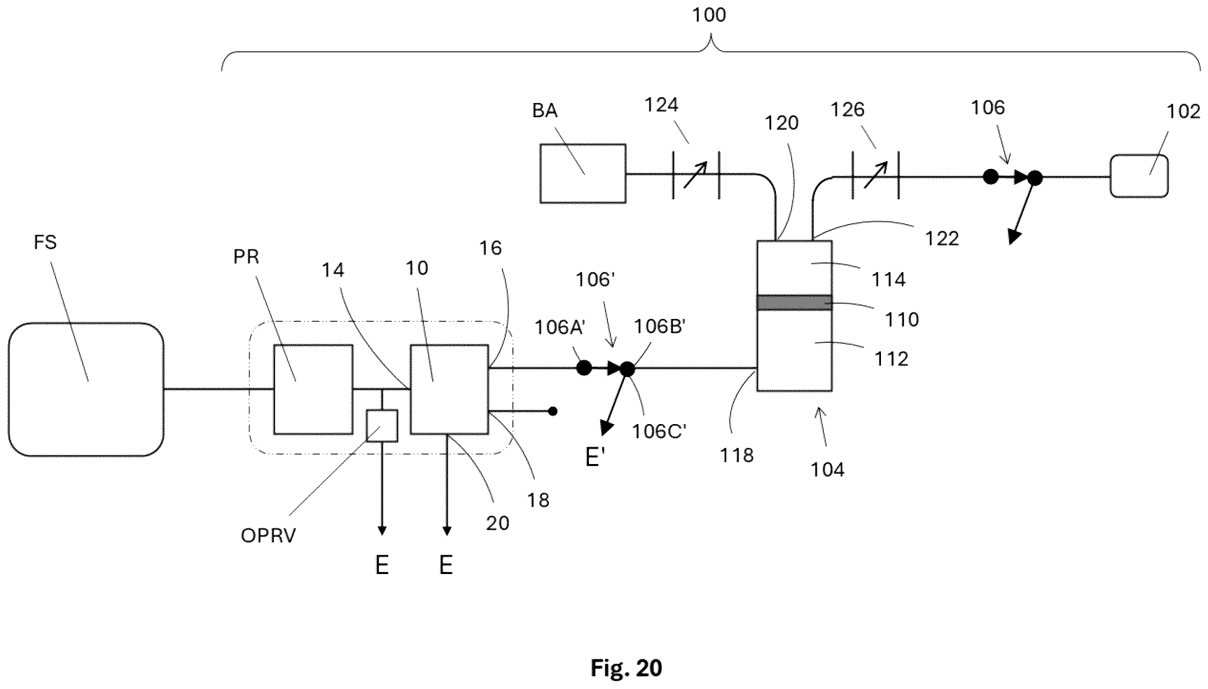

FIGS. 1-19D show an illustrative embodiment of an oscillating flow controller 10 (which may be referred to herein as an “oscillator”) and an illustrative use and operation of the same according to the present disclosure. FIGS. 20-22B show illustrative embodiments of a mechanical ventilator including the oscillator 10 and a respiratory interface connected to a bi-directional port thereof.

With reference initially to FIGS. 1-19D, the oscillator 10 includes a housing 12 defining a fluid supply port 14, a first bi-directional fluid port 16, a second bi-directional fluid port 18, a fluid exhaust port 20 (the drawings show two fluid exhaust ports 20 but only one is required), a first armature chamber 22 fluidly connected with the fluid supply port 14, a second armature chamber 24 fluidly connected with the fluid exhaust port(s) 20, a first cavity 26 fluidly connected with a first end of the second armature chamber 24, and a second cavity 28 fluidly connected with a second end of the second armature chamber 24. In embodiments, either or both of the first bi-directional port 16 and the second bi-directional port 18 may be eliminated, plugged, or otherwise deadheaded, as will be discussed further below.

As shown, the first armature chamber 22 is generally annular, and the second armature chamber 24 is stepped-cylindrical and generally concentric (or coaxial) with the first armature chamber 22. In embodiments, the first armature chamber 22 and the second armature chamber 24 may have other shapes. The first armature chamber 22 extends peripherally about at least a portion of the second armature chamber 24.

The first cavity 26 extends in a first axial direction D1 from a first end of the second armature chamber 24. The second cavity 28 extends in a second axial direction D2 from a second end of the second armature chamber 24. The first axial direction D1 is opposite the second axial direction D2.

A first fluid channel 30 fluidly connects the first armature chamber 22 with the first cavity 26. A second fluid channel 32 fluidly connects the first armature chamber 22 with the second cavity 28. A third fluid channel 34 fluidly connects the first fluid channel 30 with the first bi-directional port 16. In embodiments omitting the first bi-directional port 16, the third fluid channel 34 may be omitted, as well. A fourth fluid channel 36 fluidly connects the second fluid channel 32 with the second bi-directional port 18. In embodiments omitting the second bi-directional port 18, the fourth fluid channel 36 may be omitted, as well. A fluid exhaust channel 20C fluidly connects the second armature chamber 24 with the fluid exhaust port 20.

As shown, an optional first flow-restricting orifice 38 may be disposed in the first fluid channel 30, and an optional second flow-restricting orifice 40 may be disposed in the second fluid channel 32. The optional first Similarly, in embodiments, optional flow-restricting orifices could be installed in any fluid channel within the oscillator 10, for example, without limitation, any or all of the first and second fluid channels 30, 32, a fluid supply channel 14C and the fluid exhaust channel(s) 20C. In embodiments, any or all such flow-restricting orifices may be adjustable. Such optional flow-restricting orifices may be provided and sized as desired as factors contributing to the oscillation frequency of the flow oscillator 10, as will be discussed further below and as would be understood by one skilled in the art.

The oscillator 10 also includes a first movable pressure barrier 42 disposed in the first cavity 26 so as to divide the first cavity 26 into a first compartment 26A proximate the second armature chamber 24 and a second compartment 26B distant from the second armature chamber 24. The first movable pressure barrier 42 defines a first aperture 44 therein through which fluid may selectively flow between the first compartment 26A and the second compartment 26B, as will be discussed further below. At least a portion of the first movable pressure barrier 42 may be flexible and/or resilient.

Similarly, the oscillator 10 includes a second movable pressure barrier 52 disposed in the second cavity 28 so as to divide the second cavity 28 into a first compartment 28A proximate the second armature chamber 24 and a second compartment 28B distant from the second armature chamber 24. The second movable pressure barrier 52 defines a second aperture 54 therein through which fluid may selectively flow between the first compartment 28A and the second compartment 28B. At least a portion of the second movable pressure barrier 52 may be flexible and/or resilient.

The second compartment 26B of the first cavity 26 alone or in combination with the first fluid channel 30 and the third fluid channel 34 (if provided) comprises a first pilot chamber and may be referred to herein as the first pilot chamber 26B. Similarly, the second compartment 28B of the second cavity 28 alone or in combination with the second fluid channel 32 and the fourth fluid channel 36 (if provided) comprises a second pilot chamber and may be referred to herein as the second pilot chamber 28B.

The respective volumes of the first and second pilot chambers 26B, 28B may be sized as desired as factors contributing to the oscillation frequency of the oscillator 10, as will be discussed further below and as would be understood by one skilled in the art.

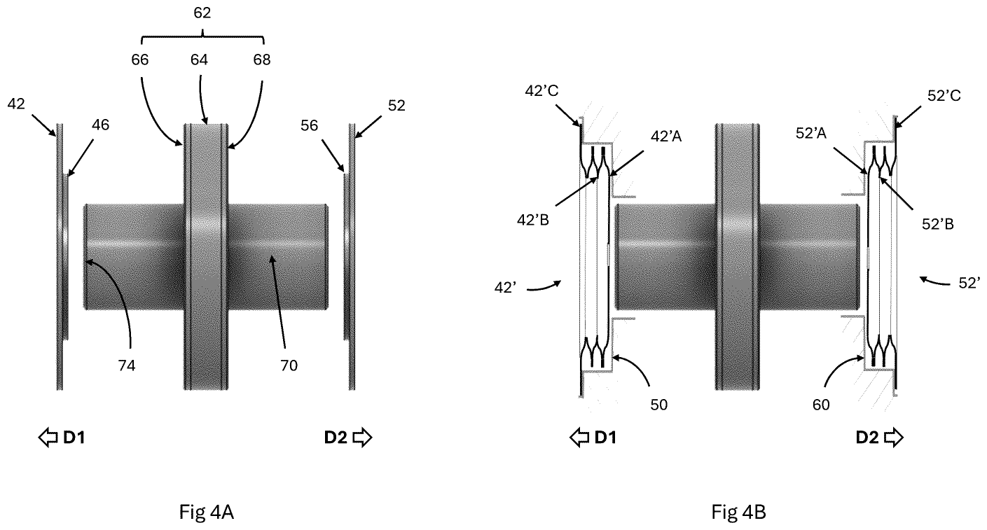

As shown generally in the drawings, the first and second movable pressure barriers 42, 52 may be embodied as first and second flexible diaphragms 42, 52. As shown in FIG. 4B, the first and second movable pressure barriers may be embodied as first and second flexible bellows 42′, 52′. In embodiments one of the first and second movable pressure barriers 42, 52 could be embodied as a flexible diaphragm, and the other could be embodied as a flexible bellows. One skilled in the art would recognize that the first and second movable pressure barriers 42, 52 could be embodied in other ways, as well.

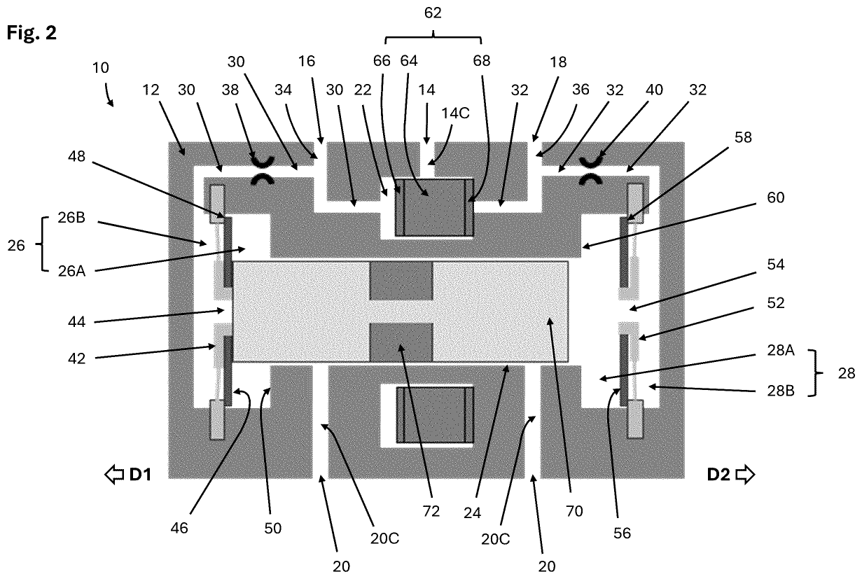

The oscillator 10 further includes a first armature 62 slidingly received within the first armature chamber 22. The first armature 62 has a first end and a second end opposite the first end. The first end of the first armature 62 faces the first direction D1, and the second end of the first armature 62 faces the second direction D2. A first permanent magnet 64 is disposed in a central region of the first armature 62 between the first and second ends thereof. The first permanent magnet 64 may be fixed to the first armature 62. The first end of the first armature 62 is configured to selectively and sealingly occlude the first fluid channel 30. For example, as may be best shown in FIG. 1, the first end of the first armature 62 is configured selectively and sealingly engage with a first wall of the first armature chamber 22 opposite the first end of the first armature and defining a corresponding end of the first fluid channel 30 to thereby occlude the first fluid channel 30. Similarly, the second end of the first armature 62 is configured to selectively and sealingly occlude the second fluid channel 32. For example, as may be best shown in FIG. 2, the second end of the first armature 62 is configured to selectively and sealingly engage with a second wall of the first armature chamber 22 opposite the second end of the first armature 62 and defining a corresponding end of the second fluid channel 32.

In embodiments, one or more seals may be provided to facilitate sealing engagement of the first armature 62 with the first and second walls of the first armature chamber 22. For example, as shown, the first end of the first armature 62 includes a first face seal 66, and the second end of the first armature 62 includes a second face seal 68. In embodiments, the first face seal 66 may instead be integrated with the housing 12 opposite the first end of the first armature 62 and be configured to selectively and sealingly engage the first end of the first armature 62. Similarly, the second face seal 68 may instead be integrated with the housing 12 opposite the second end of the first armature 62 and be configured to selectively and sealingly engage the second end of the first armature 62. In embodiments, other forms of seals (not shown) may be provided in addition to or instead of the foregoing face seals.

The first armature 62 is configured to slide axially within the first armature chamber 22 in the first and second directions D1, D2 between a first position and a second position. The first armature 62 is configured to resist or block fluid flow between the first armature chamber 22 and the first fluid channel 30, and to enable flow between the first armature chamber 22 and the second fluid channel 32 when the first armature 62 is in the first position. Also, the first armature 62 is configured to enable fluid flow between the first armature chamber 22 and the first fluid channel 30, and to resist or block fluid flow between the first armature chamber 22 and the second fluid channel 32 when the first armature 62 is in the second position. More specifically, when the first armature 62 is in the first position, the first end of the first armature 62 (and the first face seal 66 if provided) engages a wall of the first armature chamber 22 defining the corresponding end of the first fluid channel 30 and occludes the end of the first fluid channel 30, while the second end of the first armature 62 is spaced from a wall of the first armature chamber 22 defining the corresponding end of the second fluid channel 32. When the first armature 62 is in the second position, the first end of the first armature 62 is spaced from the wall of the first armature chamber 22 defining the corresponding end of the first fluid channel 30, while the second end of the first armature 62 (including the second face seal 68 if provided) engages the wall of the first armature chamber 22 defining the corresponding end of the second fluid channel 32 and occludes the second fluid channel 32. As such, the first armature 62 and the housing 12 cooperate to define a first multi-port valve. Also, as is evident from the drawings, a radial clearance between the first armature 62 and the housing 12 is sufficient to enable substantial fluid flow through the first armature chamber 22, between the first armature 62 and the housing 12 defining the first armature chamber 22.

Similarly, the oscillator 10 includes a second armature 70 slidingly received within the second armature chamber 24. The second armature 70 has a first end and a second end opposite the first end. The first end of the second armature 70 faces the first direction D1, and the second end of the second armature 70 faces the second direction D2. A second permanent magnet 72 is disposed in a central region of the second armature 70 between the first and second ends thereof. The second permanent magnet 72 may be fixed to the second armature 70. The first end of the second armature 70 is configured to selectively engage with an adjacent face of the first movable pressure barrier 42 and to thereby selectively occlude the first aperture 44. Similarly, the second end of the second armature 70 is configured to selectively engage with an adjacent face of the second movable pressure barrier 52 and to thereby selectively occlude the second aperture 54. The first end of the second armature 70 may include a first face seal 74, and the second end of the second armature 70 may include a second face seal 76. In embodiments, the first face seal 74 may be instead be integrated with the first movable pressure barrier 42 and configured to selectively and sealingly engage with first end of the second armature 70. Similarly, the second face seal 76 may be instead be integrated with the second movable pressure barrier 52 and configured to selectively and sealingly engage with the second end of the second armature 70. As such, the second armature 70, the first movable pressure barrier 42, and the second movable pressure barrier 52 cooperate to define a second multi-port valve. Also, as is evident from the drawings, a radial clearance between the second armature 70 and the housing 12 is sufficient to enable substantial fluid flow through the second armature chamber 24, between the second armature 70 and the housing 12 defining the second armature chamber 24.

The foregoing first and second multi-port valves and the housing may cooperate to define the first and second pilot chambers 26B, 28B. In embodiments including the first flow-restricting orifice 38, the first flow-restricting orifice 38 divides the first pilot chamber 26B into a first section proximate the first armature (and, therefore, the first multi-port valve) and a second section proximate the first movable pressure barrier 42 and the second armature 70 (and, therefore, the second multi-port valve). In embodiments including the second flow-restricting orifice 40, the second flow-restricting orifice 40 similarly divides the second pilot chamber 28B into a first section proximate the first armature (and, therefore, the first multi-port valve) and a second section proximate the first movable pressure barrier 42 and the second armature 70 (and, therefore, the second multi-port valve).

The second armature 70 is configured to slide axially within the second armature chamber 24 in the first and second directions D1, D2 between a first position and a second position. The second armature 70 is configured to resist or block fluid flow between the first and second compartments 28A, 28B of the second cavity 28 (and, therefore, between the second armature chamber 24 and the second pilot chamber 28B), and to enable flow between the first and second compartments 26A, 26B of the first cavity 26 (and, therefore, between the second armature chamber 24 and the first pilot chamber 26B) when the second armature 70 is in the first position. Also, the second armature 70 is configured to enable fluid flow between the first and second compartments 28A, 28B of the second cavity 28 (and, therefore, between the second armature chamber 24 and the second pilot chamber 28B), and to resist or block flow between the first and second compartments 26A, 26B of the first cavity 26 (and, therefore, between the second armature chamber 24 and the first pilot chamber 26B) when the second armature 70 is in the second position. More specifically, when the second armature 70 is in the first position, the second end of the second armature 70 (and the second face seal 76 if provided) engages the adjacent face of the second movable pressure barrier 52 and occludes the second aperture 54, while the first end of the second armature 70 is spaced from the first movable pressure barrier 42 and the first aperture 44. When the second armature 70 is in the second position, the second end of the second armature 70 is spaced from the second movable pressure barrier 52 and the second aperture 54, while the first end of the second armature 70 (including the first face seal 74 if provided) engages the adjacent face of the first movable pressure barrier 42 and occludes the first aperture 44.

In embodiments, the oscillator 10 may include a first travel limiter configured to restrict displacement of the first movable pressure barrier 42 relative to one or both of the first movable pressure barrier 42 and the housing 12. For example, and with reference to FIG. 4A, wherein the first movable pressure barrier 42 is shown as a first flexible diaphragm 42, the first travel limiter may be embodied as a first flange 46 adhered to or otherwise integrated with a central portion of the first flexible diaphragm 42 proximate the first aperture 44. The first flange 46 may be configured to engage a land 48 defined by a peripheral portion of the first flexible diaphragm 42 radially outward from the central portion and the first aperture 44 of the first flexible diaphragm 42 when the first flexible diaphragm 42 is displaced in the first direction D1, thereby limiting displacement of the first flexible diaphragm 42 in the first direction D1. Also, the housing 12 may define a land 50 configured to engage with the first flange 46 when the first flexible diaphragm 42 is displaced in the second direction D2, thereby limiting displacement of the first flexible diaphragm 42 in the second direction D2.

Similarly, the oscillator 10 may include a second travel limiter configured to restrict displacement of the second movable pressure barrier 52 relative to one or both of the second movable pressure barrier 52 and the housing 12. For example, and with continued reference to FIG. 4A, wherein the second movable pressure barrier 52 is shown as a second flexible diaphragm 52, the second travel limiter may be embodied as a second flange 56 adhered to or otherwise integrated with a central portion of the second movable pressure barrier 52 proximate the second aperture 54. The second flange 56 may be configured to engage a land 58 defined by a peripheral portion of the second movable pressure barrier 52 radially outward from the central portion and the second aperture 54 of the second movable pressure barrier 52 when the second movable pressure barrier 52 is displaced in the second direction D2, thereby limiting displacement of the second movable pressure barrier 52 in the second direction D2. Also, the housing 12 may define a land 60 configured to engage with the second flange 56 when the second movable pressure barrier 52 is displaced in the first direction D1, thereby limiting displacement of the second movable pressure barrier 52 in the first direction D1.

In embodiments wherein one or both of the first and second pressure barriers 42, 52 are first and second flexible bellows 42′, 52′, for example, as shown in FIG. 4B, respective portions of the respective flexible bellows 42′, 52′ may inherently function as first and second travel limiters in the directions of extension and compression of the respective flexible bellows 42′, 52′, as would be recognized by one skilled in the art. For example, respective innermost segments 42′A, 52′A of the first and second flexible bellows 42′, 52′ may be configured to contact the respective first and second lands 50, 60 when the first and second flexible bellows 42′, 52′ are extended and thus limit the extension thereof. Also, the respective innermost segments 42′A, 52′A of the first and second flexible bellows 42′, 52′ and intervening segments 42′B, 52′B of the first and second flexible bellows 42′, 52′ may be configured to stack up against respective outermost segments 42′C, 52′C of the first and second flexible bellows 42′, 52′ when the first and second flexible bellows 42′, 52′ are compressed. As shown in FIG. 4B, the direction of extension of the first flexible bellows 42′ is the second direction D2, and the direction of compression of the first flexible bellows 42′ is the first direction D1. As also shown in FIG. 4B, the direction of extension of the second flexible bellows 52′ is the first direction D1, and the direction of compression of the second flexible bellows 52′ is the second direction D2.

As mentioned above, the first armature 62 includes a first magnet 64, and the second armature 70 includes a second magnet 72. As shown, the first magnet 64 surrounds and is generally coaxial with the second magnet 72. Also, as best shown in, for example, FIGS. 1 and 2, respectively, the first magnet 64 is axially offset from the second magnet 72 in the first direction D1 when the first armature 62 and the second armature 70 are in their respective first positions, and the first magnet 64 is axially offset from the second magnet 72 in the second direction D2 when the first armature 62 and the second armature 70 are in their respective second positions. More specifically, a magnetic center of the first magnet 64 is axially offset from a magnetic center of the second magnet 70 in the first direction D1 when the first and second armatures 62, 70 are in their respective first positions, and the magnetic center of the first magnet 64 is axially offset from the magnetic center of the second magnet 70 in the second direction D2 when the first and second armatures 62, 70 are in their respective second positions.

As suggested above, the first and second magnets 62, 70 cooperate to define a biasing mechanism configured to simultaneously bias both the first and second armatures 62, 70 (and, therefore the first and second multi-port valves they respective define) toward their respective first positions or their respective second positions. More specifically, the first and second magnets 64, 72 are configured so that a magnetic field between the first and second magnets 64, 72 causes the first and second magnets 64, 72 to repel each other at least axially. The strength of the magnetic field and, therefore, the repulsive force, is greatest when the magnetic centers of the first and second magnets 62, 70 are nearest to each other. Conversely, the strength of the magnetic field and, therefore, the repulsive force, is lowest when the magnetic centers of the first and second magnets 62, 70 are farthest from each other. As such, the magnetic field biases the first and second armatures 62, 70 toward their respective first positions when the first magnet 64 is axially offset from the second magnet 72 in the first direction D1. Similarly, the magnetic field biases the first and second armatures 62, 70 toward either of their respective second positions when the first magnet 64 is axially offset from the second magnet 72 in the second direction D2. Thus, in the absence of other forces acting on the first and second armatures 62, 70, the first and second armatures 62, 70 are stable when both of the first and second armatures 62, 70 are in either their respective first positions or their respective second positions. The respective field strengths of the first and second magnets 64, 72 may be selected as desired as factors contributing to the oscillation frequency of the flow oscillator 10, as would be recognized by one skilled in the art. As shown, the first and second magnets 64, 72 are located at generally central portions of the first and second armatures 62, 70, respectively. In embodiments, the first and second magnets 64, 72 could be located elsewhere with respect to the first and second armatures 62, 70, respectively, with the magnetic centers of the first and second magnets 64, 72 configured to interact with each other as described above. For example, the first and second magnets 64, 72 could be located proximate the respective first ends or second ends of the first and second armatures 62, 70. In embodiments, the foregoing magnetic biasing mechanism could be replaced with another magnetic biasing mechanism or a non-magnetic biasing mechanism configured to simultaneously bias the first and second armatures 62, 70 towards their respective first positions or their respective second positions.

As mentioned above, the volumes of the first and second pilot chambers as described above, the selection of optional orifices in and or all of the first, second, third, and fourth fluid channels 30, 32, 34, 36 and the exhaust channel(s) 20C, and the magnetic field strength between the first and second magnets 64, 72 are factors that contribute to the oscillation frequency of the oscillator 10. Other factors may contribute to the oscillation frequency of the oscillator 10, including without limitation: the respective sizes of the fluid supply port 14, the first and second bi-directional ports 16, 18, and the fluid exhaust port(s) 20; the respective sizes of the first and second apertures 44, 54; the respective clearances between the second armature 70 and the first and second apertures 44, 54; the clearance between the second armature 70 and the portion of the housing 12 defining the second armature chamber 24; the respective clearances between the first armature 62 and the portions of the housing 12 defining the corresponding adjacent ends of the first and second flow channels 30, 32; and the movable surface area of the first and second movable pressure barriers 42, 52. One skilled in the art would understand how to select at least the foregoing components or features in order to achieve a desired oscillation frequency of the oscillator 10, as will be discussed further below.

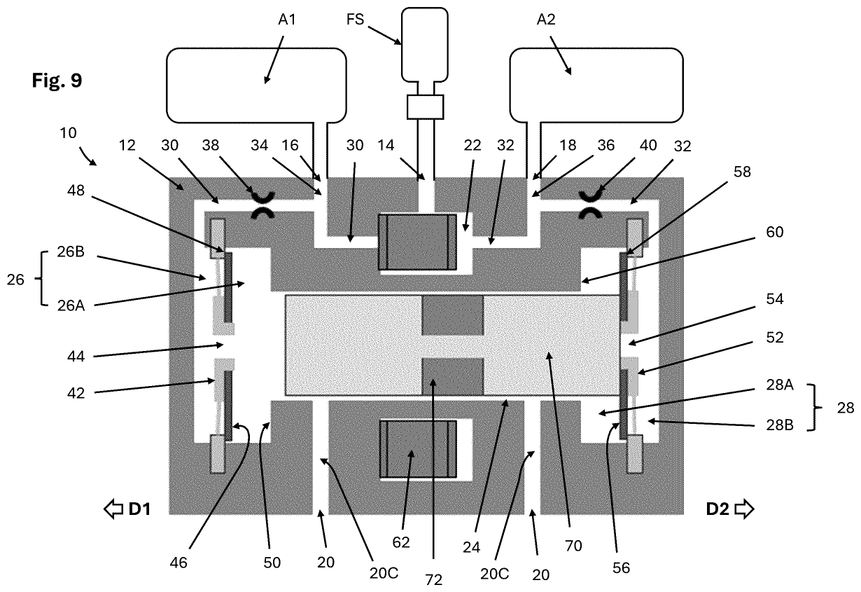

As best shown in FIGS. 9 and 10, the oscillator 10 is configured for use in connection with a source of pressurized fluid FS in fluid communication with the fluid supply port 14, a first external accumulator A1 in fluid communication with the first bi-directional fluid port 16, and a second external accumulator A2 in fluid communication with the second bi-directional fluid port 18. The pressurized fluid may be a liquid or a gas, for example, air. The first and second external accumulators A1, A2 may be, without limitation, first and second selectively inflatable compartments of a therapeutic support surface overlay (not shown). One skilled in the art would recognize that the respective volumes and internal flow characteristics of the first and second external accumulators A1, A2, as well the respective volumes and flow characteristics of fluid conduits connecting the first and second external accumulators A1, A2, respectively, to the first and second bi-directional ports 16, 18 are factors that may contribute to the oscillation frequency of the flow oscillator 10.

FIG. 9 shows the oscillator 10 in a first, stable state, with no fluid flowing from the source of pressurized fluid FS and with the first and second external accumulators A1, A2 at an ambient pressure. In this first, stable state, the first and second armatures 62, 70 are in their respective first positions and are biased to their respective first positions by the magnetic biasing force between the first and second magnets 64, 72. As such, the first armature 62 enables fluid flow from the first armature chamber 22 into the second fluid channel 32 and, therefore, to the second pilot chamber 28B, the fourth fluid channel 36, and the second bi-directional fluid port 18, while blocking fluid flow from the first armature chamber 22 into the first fluid channel 30 and, therefore, to the first pilot chamber 26B, the third fluid channel 34, and the first bi-directional fluid port 16. Also, the second armature 70 enables flow from the first fluid channel 30 through the first aperture 44 of the first movable pressure barrier 42, while resisting or blocking flow from the second fluid channel 32 through the second aperture 54 of the second movable pressure barrier 52. Thus, in this first, stable state, the interior portion of the housing 12 including the second armature chamber 24, the first pilot chamber 26B, and the first and third fluid channels 30, 34 are vented via the exhaust port 20 to an environment, for example, the atmosphere, surrounding the oscillator 10 and thus are at an ambient pressure.

FIG. 10 shows the oscillator 10 in a second, stable state, with no fluid flowing from the source of pressurized fluid FS and with the first and second external accumulators A1, A2 at an ambient pressure. In this second, stable state, the first and second armatures 62, 70 are in their respective second positions and are biased to their respective second positions by the magnetic biasing force between the first and second magnets 64, 72. As such, the first armature 62 blocks fluid flow from the first armature chamber 22 into the second fluid channel 32 and, therefore, to the second pilot chamber 28B, the fourth fluid channel 36, and the second bi-directional fluid port 18, while enabling fluid flow from the first armature chamber 22 into the first fluid channel 30 and, therefore, to the first pilot chamber 26B, the third fluid channel 34, and the first bi-directional fluid port 16. Also, the second armature 70 blocks flow from the first fluid channel 30 through the first aperture 44 of the first movable pressure barrier 42, while enabling flow from the second fluid channel 32 through the second aperture 54 of the second movable pressure barrier 52. Thus, in this second, stable state, the interior portion of the housing 12 including the second armature chamber 24, the second pilot chamber 28B, and the second and fourth fluid channels 32, 36 are vented via the exhaust port 20 to an environment, for example, the atmosphere, surrounding the oscillator 10 and thus is at an ambient pressure.

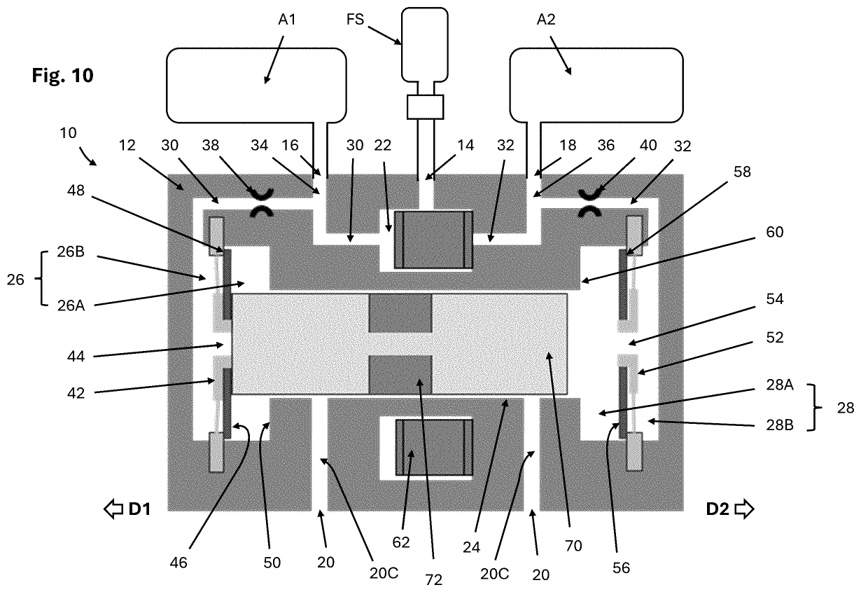

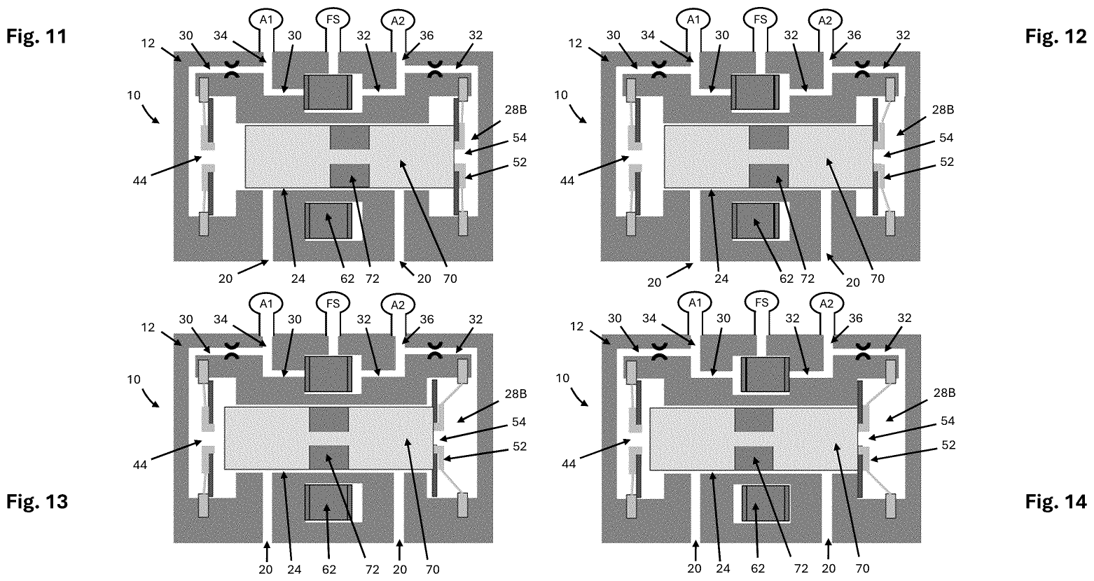

FIGS. 11-18 show the oscillator 10 connected to and receiving pressurized fluid from the source of pressurized fluid FS and selectively directing the pressurized fluid to or relieving the pressurized fluid from the first and second external accumulators A1, A2 in various operational states.

FIG. 11 shows the oscillator 10 in a first initial pressurization state, wherein the first and second armatures 62, 70 are in their respective first positions, as discussed further above and shown in FIG. 9. In this state, the oscillator 10 receives pressurized fluid from the source of pressurized fluid FS via the fluid supply port 14 and directs the pressurized fluid to: (i) the second pilot chamber 28B via the first armature chamber 22 and the second fluid channel 32; and (ii) the second external accumulator A2 via the first armature chamber 22, the second and fourth fluid channels 32, 36 and the second bi-directional fluid port 18. The foregoing flow of pressurized fluid causes the fluid pressures within the second external accumulator A2 and the second pilot chamber 28B to increase. One skilled in the art would recognize that the second and fourth channels 32, 36, and the second flow-restricting orifice 40 (if provided) may be configured so that the pressurized fluid enters the second pilot chamber 28B at a similar rate or at a substantially different rate (for example, more slowly) than that at which it enters the second external accumulator A2 and, therefore, that the fluid pressure within the second pilot chamber 28B increases at a similar rate or a substantially different rate than does the fluid pressure within the second external accumulator A2. Also, in the first initial pressurization state of FIG. 11, the first armature 62 disables flow from the first armature chamber 22 to and downstream of the first fluid channel 30 and, therefore, disables flow of pressurized fluid from the source of pressurized fluid FS to the first accumulator A1 and the first pilot chamber 26B. Further, the second armature 70 blocks fluid flow through the second aperture 54 and enables fluid flow through the first aperture 44, thereby enabling flow of pressurized fluid from the first external accumulator A1 through the fluid exhaust port(s) 20 via the third fluid channel 34, the first fluid channel 30, the first pilot chamber 26B, the second armature chamber 24 and the fluid exhaust channel(s) 20C.

FIG. 12 shows the oscillator 10 in a first intermediate pressurization state similar to the first initial pressurization state of FIG. 11 wherein the first armature 62 remains in its first position, but wherein increasing fluid pressure within the second chamber 28B has caused the second movable pressure barrier 52 to move in the first direction D1 and thereby displace the second armature 70 in the first direction D1. Consequently, the magnetic center of the second magnet 72 has been displaced in the first direction D1 toward, but not to, the magnetic center of the first magnet 64. Therefore, the repulsive magnetic force between the first magnet 64 and the second magnet 72 continues to bias the first magnet 64 (and thus the first armature 62) in the first direction D1, and to bias the second magnet 72 (and thus the second armature 70) in the second direction D2. (Because the first and second magnets 64, 72 are closer together in the first intermediate pressurization state of FIG. 12 compared to the first initial pressurization state of FIG. 11, the repulsive magnetic biasing force between the first and second magnets 64, 72, is greater in the first intermediate pressurization state FIG. 12 state than in the first initial pressurization state of FIG. 11, thus increasing the sealing pressure at the interface between the second end of the second armature 70 and the second movable pressure barrier 52.) As such, the alignment of fluid paths within the oscillator 10 remains as in the first initial pressurization state of FIG. 11. It follows that pressurized fluid continues to flow into the second external accumulator A2 and into the second pilot chamber 28B, the first armature 62 continues to disable flow of pressurized fluid from the source of pressurized fluid FS to and downstream of the first fluid channel 30, and the second armature 70 continues to enable fluid flow from the first external accumulator A1 to the environment surrounding the oscillator 10.

FIG. 13 shows the oscillator 10 in a second intermediate pressurization state similar to the first intermediate pressurization state of FIG. 12 wherein the first armature 62 remains in its first position, but wherein increasing fluid pressure within the second pilot chamber 28B has caused the second movable pressure barrier 52 to move further in the first direction D1 and thereby displace the second armature 70 further in the first direction D1 than in the first intermediate pressurization state of FIG. 12. Consequently, the magnetic center of the second magnet 72 has been displaced in the first direction D1 substantially to, but not beyond, the magnetic center of the first magnet 64. (Because the first and second magnets 64, 72 are closer together in the second intermediate pressurization state of FIG. 12 compared to the first intermediate pressurization state of FIG. 12, the magnetic biasing force between the first and second magnets 64, 72, is even greater in the second intermediate pressurization state FIG. 12 state than in the first intermediate pressurization state of FIG. 12, thus further increasing the sealing pressure at the interface between the second end of the second armature 70 and the second movable pressure barrier 52.) In this state, the magnetic biasing force between the first magnet 64 and the second magnet 72 destabilizes the first magnet 64 with respect to the second magnet 72. Notwithstanding, in the second intermediate pressurization state of FIG. 13, the alignment of fluid paths with the oscillator 10 remains as in the first initial pressurization state of FIG. 11. As such, pressurized fluid continues to flow into the second external accumulator A2 and into the second pilot chamber 28B, the first armature 62 continues to disable flow of pressurized fluid from the source of pressurized fluid FS to and downstream of the first fluid channel 30, and the second armature 70 continues to enable fluid flow from the first external accumulator A1 to the environment surrounding the oscillator 10.

FIG. 14 shows the oscillator 10 in a first transition state wherein the oscillator 10 transitions between the states shown in FIGS. 9 and 10, respectively. More specifically, FIG. 14 shows the oscillator 10 in a state wherein continued increasing fluid pressure within the second pilot chamber 28B has caused the second movable pressure barrier 52 to move still further in the first direction D1 (to the greatest extent permitted by the interaction of the second 56 with the land 60) and to thereby displace the second armature 70 still further in the first direction D1 than in the second intermediate pressurization state of FIG. 13. As shown in FIG. 14, the magnetic center of the second magnet 72 has been displaced in the first direction D1 beyond the magnetic center of the first magnet 64. As such, the direction of the magnetic biasing force between the first magnet 64 and the second magnet 72 has been reversed so that the magnetic biasing force biases the first magnet 64 in the second direction D2, and so that it biases the second magnet 72 in the first direction D1. Indeed, FIG. 14 shows the first armature 62 having been biased to its second position as shown in FIG. 10, and it shows the second armature 70 being biased toward, but not having been biased to, its second position.

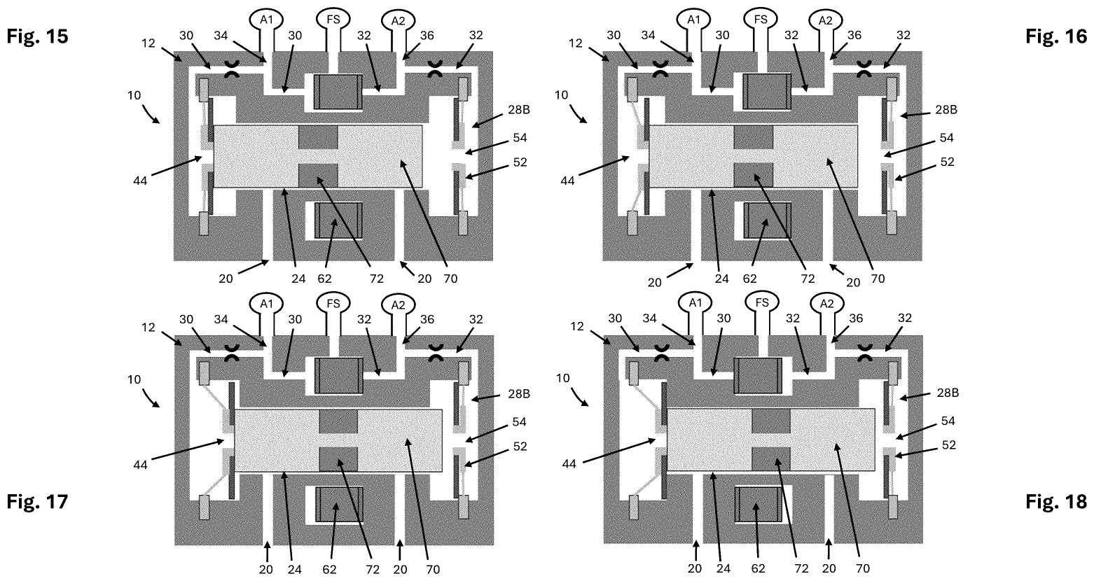

FIG. 15 shows the oscillator 10 in a second initial pressurization state, wherein the first and second armatures 62, 70 are in their respective second positions, as discussed further above and shown in FIG. 10. In this state, the oscillator 10 receives pressurized fluid from the source of pressurized fluid FS via the fluid supply port 14 and directs the pressurized fluid to: (i) the first pilot chamber 26B via the first armature chamber 22 and the first fluid channel 30; and (ii) the first external accumulator A1 via the first armature chamber 22, the first fluid channel 30, the third fluid channel 34, and the first bi-directional fluid port 16. The foregoing flow of pressurized fluid causes the fluid pressures within the first external accumulator A1 and the first pilot chamber 26B to increase. One skilled in the art would recognize that the first and third fluid channels 30, 34, and the first flow-restricting orifice 38 (if provided) may be configured so that the pressurized fluid enters the first pilot chamber 26B at a similar rate or at a substantially different rate (for example, more slowly) than it enters the first external accumulator A1 and, therefore, that the fluid pressure within the first pilot chamber 26B increases at a similar rate or a substantially different rate than does the fluid pressure within the first external accumulator A1. Also, in the second initial pressurization state of FIG. 15, the first armature 62 disables flow from the first armature chamber 22 to the second fluid channel 32 and, therefore, disables flow of pressurized fluid from the source of pressurized fluid FS to the second external accumulator A2 and the second pilot chamber 28B. Further, the second armature 70 blocks fluid flow through the first aperture 44 and enables fluid flow through the second aperture 54, thereby enabling flow of pressurized fluid from the second external accumulator A2 through the fluid exhaust port(s) 20 via the fourth fluid channel 36, the second fluid channel 32, the second pilot chamber 28, the second armature chamber 24 and the fluid exhaust channel(s) 20C.

FIG. 16 shows the oscillator 10 in a third intermediate pressurization state similar to the second initial pressurization state of FIG. 11 wherein the first armature 62 remains in its second position, but wherein increasing fluid pressure within the first pilot chamber 26B has caused the first movable pressure barrier 42 to move in the second direction D2 and thereby displace the second armature 70 in the second direction D2. Consequently, the magnetic center of the second magnet 72 has been displaced in the second direction D2 toward, but not to, the magnetic center of the first magnet 64. Therefore, the magnetic force between the first magnet 64 and the second magnet 72 continues to bias the first magnet 64 (and thus the first armature 62) in the second direction D2, and to bias the second magnet 72 (and thus the second armature 70) in the first direction D1. (Because the first and second magnets 64, 72 are closer together in the third intermediate pressurization state of FIG. 16 compared to the second initial pressurization state of FIG. 15, the magnetic biasing force between the first and second magnets 64, 72, is greater in the third intermediate pressurization state FIG. 16 state than in the second initial pressurization state of FIG. 15, thus increasing the sealing pressure at the interface between the second end of the second armature 70 and the second movable pressure barrier 52.) As such, the alignment of fluid paths within the oscillator 10 remains as in the second initial pressurization state of FIG. 15. It follows that pressurized fluid continues to flow into the first external accumulator A1 and into the first pilot chamber 26B, the first armature 62 continues to disable flow of pressurized fluid from the source of pressurized fluid FS to and downstream of the second fluid channel 32, and the second armature 70 continues to enable fluid flow from the second external accumulator A2 to the environment surrounding the oscillator 10.

FIG. 17 shows the oscillator 10 in a fourth intermediate pressurization state similar to the third intermediate pressurization state of FIG. 16 wherein the first armature 62 remains in its second position, but wherein increasing fluid pressure within the first pilot chamber 26B has caused the first movable pressure barrier 42 to move further in the second direction D2 and thereby displace the second armature 70 further in the second direction D2 than in the third intermediate pressurization state of FIG. 16. Consequently, the magnetic center of the second magnet 72 has been displaced in the second direction D2 substantially to, but not beyond, the magnetic center of the first magnet 64. (Because the first and second magnets 64, 72 are closer together in the fourth intermediate pressurization state of FIG. 17 compared to the third intermediate pressurization state of FIG. 16, the magnetic biasing force between the first and second magnets 64, 72, is even greater in the fourth intermediate pressurization state FIG. 17 than in the third intermediate pressurization state of FIG. 16, thus further increasing the sealing pressure at the interface between the second end of the second armature 70 and the second movable pressure barrier 52.) In this state, the magnetic biasing force between the first magnet 64 and the second magnet 72 destabilizes the first magnet 64 with respect to the second magnet 72. Notwithstanding, in the fourth intermediate pressurization state of FIG. 17, the alignment of fluid paths with the oscillator 10 remains as in the second initial pressurization state of FIG. 15. As such, pressurized fluid continues to flow into the first external accumulator A1 and into the first pilot chamber 26B, the armature 62 continues to disable flow of pressurized fluid from the source of pressurized fluid FS to and downstream of the second fluid channel 32, and the second armature 70 continues to enable fluid flow from the second external accumulator A2 to the environment surrounding the oscillator 10.

FIG. 18 shows the oscillator 10 in a second transition state wherein the oscillator 10 transitions between the states shown in FIGS. 10 and 9, respectively. More specifically, FIG. 18 shows the oscillator 10 in a state wherein continued increasing fluid pressure within the first pilot chamber 26B has caused the first movable pressure barrier 42 to move still further in the second direction D2 (to the greatest extent permitted by the interaction of the first travel limiter 46 with the land 50) and to thereby displace the second armature 70 still further in the second direction D2 than in the fourth intermediate pressurization state of FIG. 17. As shown in FIG. 18, the magnetic center of the second magnet 72 has been displaced in the second direction D2 beyond the magnetic center of the first magnet 64. As such, the direction of the magnetic biasing force between the first magnet 64 and the second magnet 72 has been reversed so that the magnetic biasing force biases the first magnet 64 in the first direction D1, and so that it biases the second magnet 72 in the second direction D2. Indeed, FIG. 18 shows the first armature 62 having been biased to its first position as shown in FIG. 9, and it shows the second armature 70 being biased toward, but not having been biased to, its second position. Following the second transition state of FIG. 18, the oscillator 10 returns to the first initial pressurization state of FIG. 11.

One skilled in the art would understand how to set the frequency of oscillation of the oscillator 10 as shown, for example, in FIGS. 11-18 as a function of various factors, for example, the fluid supply pressure, the sizes of the first and second accumulators A1, A2, and the movable surface area, the thickness, and the material of the first and second movable pressure barriers 42, 52, among others. The frequency of oscillation could be symmetric (the dwell time in position 1 may be the same as the dwell time in position 2) or asymmetric (the dwell time in position 1 may be different than the dwell time in position 2).

Also, one skilled in the art would recognize that the pressurization period and frequency for the first external accumulator A1 is a function of the rate of pressurization of the first pilot chamber 26B, and that the pressurization period and frequency for the second external accumulator A2 is a function of the rate of pressurization of the second pilot chamber 28B, among other factors. As such, one skilled in the art would recognize that the pressurization period and frequency of the first and second external accumulators A1, A2 may be selected as desired and that the pressurization period and frequency of the first external accumulator A1 may be the same as or different from the pressurization period and frequency of the second external accumulator A2. One skilled in the art would recognize that the oscillator 10 could be configured to charge either or both of the first and second external accumulators A1, A2 to pressures higher than the first and/or second pilot chamber 26B, 28B transition pressures by appropriate selection of, for example without limitation, the magnetic field strength(s) of the first and second magnets 64, 72 (and thus the biasing force between the first and second magnets 64, 72), the material and movable surface area of the first and/or second movable pressure barriers 42, 52, and the size of one or more internal fluid channels and and/or flow-restricting orifices within the oscillator 10.

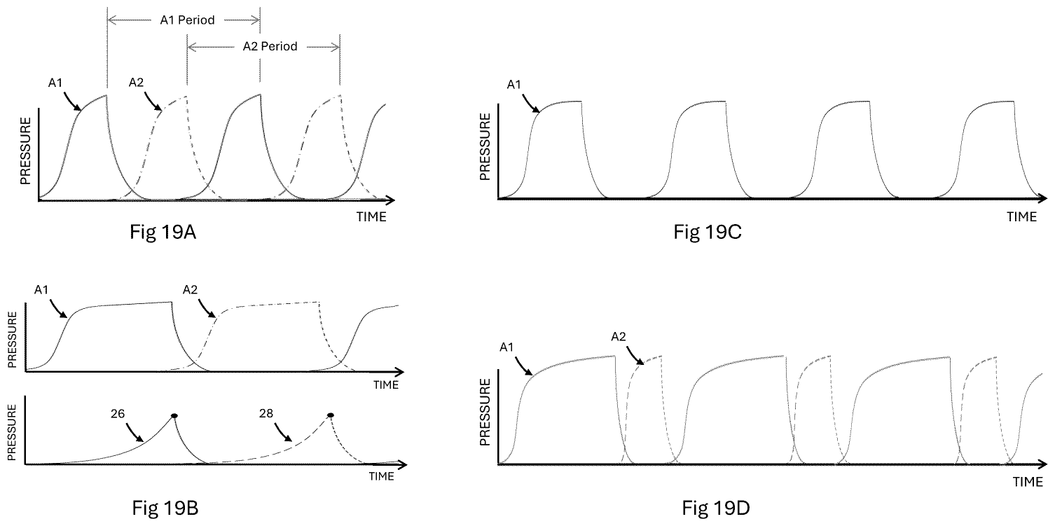

FIGS. 19A-19D are timing diagrams showing illustrative pressurization and venting of ones of the first and second external accumulators A1, A2 resulting from illustrative oscillations of the oscillator 10 as functions of time. More specifically, FIG. 19A shows timing of pressurization and venting of the first and second external accumulators A1, A2 as a function of time according to a first illustrative embodiment further to corresponding oscillation of the oscillator 10, wherein the pressurization period and frequency of the first external accumulator A1 is the same as the pressurization period and frequency of the second external accumulator A2. FIG. 19B shows timing of pressurization and venting of the first and second external accumulators A1, A2 as a function of time according to a second illustrative embodiment further to corresponding oscillation of the oscillator 10, wherein the pressurization period and frequency of the first external accumulator A1 is the same as the pressurization period and frequency of the second external accumulator A2. FIG. 19B also shows timing of corresponding pressurization and venting of the first and second pilot chambers 26B, 28B of the oscillator 10 according to the second illustrative embodiment. FIG. 19C shows timing of pressurization and venting of the first external accumulator A1 as a function of time according to a third illustrative embodiment further to corresponding oscillation of the oscillator 10. In the FIG. 19C embodiment, the second external accumulator A2 is omitted and the second bi-directional fluid port 18 is omitted or plugged. FIG. 19D shows timing of pressurization and venting of the first and second external accumulators A1, A2 as a function of time according to a fourth illustrative embodiment further to corresponding oscillation of the oscillator 10, wherein the pressurization period and frequency of the first external accumulator A1 is different from the pressurization period and frequency of the second external accumulator A2.

As mentioned above, either or both of the first and second bi-directional ports 16, 18 may be omitted or plugged. In such embodiments, the respective one or ones of the first and second external accumulators A1, A2 also would be omitted. In such embodiments, the operation of the flow oscillator 10 may similar to that described above, except that the flow control valve would not communicate fluid with the omitted one or ones of the first and second external accumulators A1, A2, and the structures of the omitted or plugged one or ones of the first and second bi-directional ports 16, 18 and the omitted one or ones of the first and second external accumulators A1, A2 (and associated fluid conduits) would not be factors contributing to the oscillation frequency of the oscillator 10. Accordingly, in such embodiments, the size of the pilot chambers corresponding to the omitted or plugged one(s) of the first and second bi-directional ports 16, 18, as discussed further above, and the size(s) of the flow restrictors corresponding to the omitted or plugged one(s) of the first and second bi-directional ports 16, 18 may be particularly relevant to (and may be the predominant factors in) achieving desired oscillation frequency characteristics for the oscillator 10, as would be understood by one skilled in the art.

As also mentioned above, either or both of the first and second bi-directional ports 16, 18 may deadheaded, for example without limitation, by plugging a respective one or ones of fluid conduits connected thereto, external to the housing 12. In such embodiments, the structures of the deadheaded one or ones of the first and second bi-directional ports 16, 18 and the fluid conduits connected thereto may remain factors contributing to the oscillation frequency of the oscillator 10. Nevertheless, in such embodiments, the size of the pilot chamber 26B, 28B corresponding to the omitted or plugged one(s) of the first and second bi-directional ports 16, 18 and the size(s) of the flow restrictors corresponding to the omitted or plugged one(s) of the first and second bi-directional ports 16, 18 may be particularly relevant to (and may be the predominant factors in) achieving desired oscillation frequency characteristics for the oscillator 10, as discussed further above, and as would be understood by one skilled in the art.

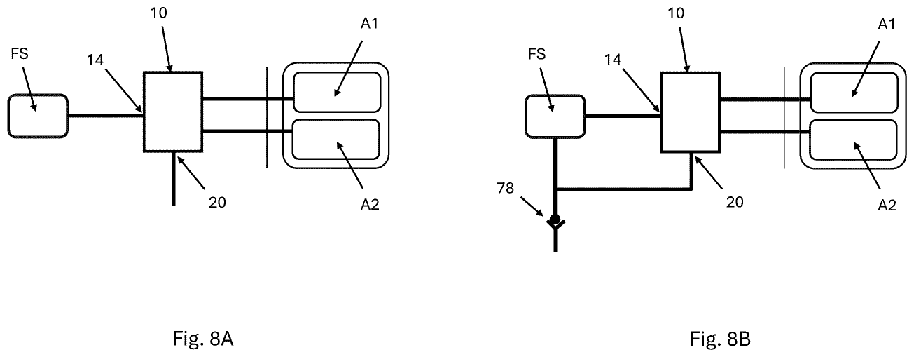

The foregoing description of operation of the oscillator 10 is directed to an embodiment wherein the exhaust port 20 is fluidly coupled to an environment surrounding the oscillator 10, and wherein the environment may be the atmosphere at ambient pressure. In embodiments, the exhaust port 20 may by fluidly coupled to an environment other than the atmosphere, wherein the environment may be at a pressure other than ambient pressure. For example, as shown in FIG. 8B, the exhaust port 20 may be coupled to an environment at a pressure greater than ambient pressure or less than ambient pressure, for example, a vacuum. In an embodiment, the source of pressurized fluid FS could be a fluid pump, for example a pneumatic pump, having a fluid inlet and a fluid outlet. The fluid inlet port 14 of the oscillator 10 could be fluidly coupled to the fluid outlet of the fluid pump, and the exhaust port 20 of the oscillator 10 could be fluidly coupled to the fluid inlet of the fluid pump. Thus, the fluid pump could draw a vacuum on the exhaust port 20 of the oscillator 10, while simultaneously providing pressurized fluid to the fluid inlet port 14 of the oscillator 10. Such an arrangement may facilitate evacuation of one or more external accumulators, for example, one or both of the first and second external accumulators A1, A2, connected to one or both of the bi-directional ports 16, 18 during operation of the oscillator 10. A vacuum breaker, for example, a calibrated check valve, could be provided in fluid communication with the pump inlet to provide make up fluid to the pump inlet. In any event, the pressure at the exhaust port 20 would be less than the pressure of the fluid provided to the fluid inlet port 14.

FIGS. 20-22B show illustrative embodiments of a mechanical ventilators including the oscillator 10 and a respiratory interface connected to the first bi-directional port 16 thereof in fluid communication therewith. The respiratory interface may be embodied in any suitable form configured to provide pressurized breathing air to a user's lung or lungs or otherwise to the user's respiratory system. For example without limitation, the respiratory interface may be embodied as a positive pressure face mask (configured to provide pressurized breathing air to a user's mouth or nose or both) or a tracheal tube configured for intubation into a user, as will be discussed further below. Such a tracheal tube may be, for example without limitation, an endotracheal tube, a tracheostomy tube, or a nasotracheal tube.

FIG. 20 shows schematically a mechanical ventilator 100 including the oscillator 10, a respiratory interface 102, an accumulator 104, a directional control valve 106, an optional directional control valve 106′, an optional pressure regulator PR, and an optional overpressure relief valve OPRV. FIG. 20 shows the respiratory interface 102, the accumulator 104, the directional control valve 106, and the optional directional control valve 106′ fluidly coupled (directly or indirectly) to the first bi-directional port 16 of the oscillator 10, with the second bidirectional port 18 eliminated, blocked, plugged, or otherwise deadheaded. In embodiments, the accumulator 104, the directional control valve 106, and the optional directional control valve 106′ may instead be fluidly coupled (directly or indirectly) to the second bi-directional port 18 of the oscillator 10, with the first bidirectional port 16 eliminated, blocked, plugged, or otherwise deadheaded. In embodiments, the accumulator 104, the directional control valve 106, and the optional directional control valve 106′ may be fluidly coupled (directly or indirectly) to the first bi-directional port 16 of the oscillator 10, and a second respiratory interface 102, a second accumulator 104, a second directional control valve 106, and a second optional directional control valve 106′ may be fluidly coupled (directly or indirectly) to the second bi-directional port 18 of the oscillator 10.

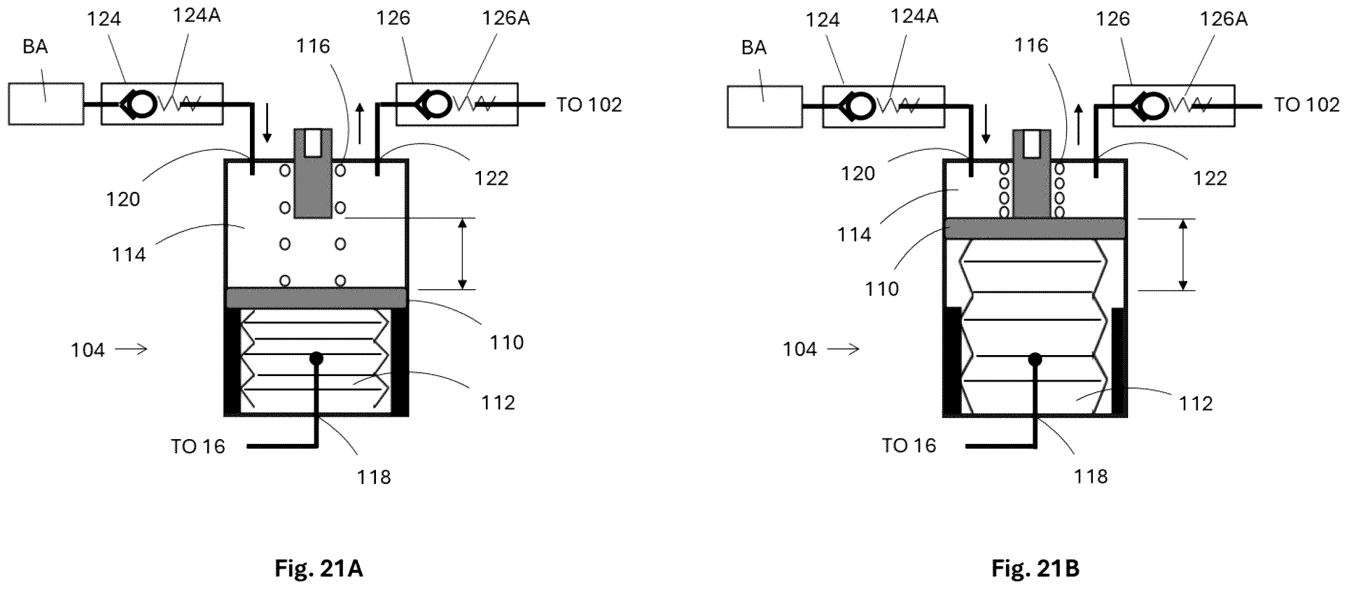

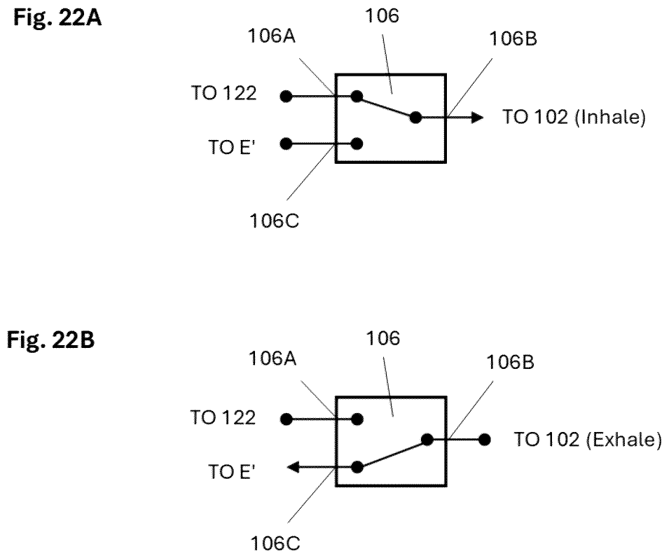

FIGS. 21A and 21B, respectively, show the accumulator 104 in greater detail and in first and second operational states, as will be discussed further below. FIGS. 22A and 22B, respectively, show schematically internal flow paths of the directional control valve 106 schematically in first and second operational states. The optional directional control valve 106′ may be similarly configured.

The accumulator 104 is shown as having a housing 108 and a diaphragm 110 in displaceable, sealing engagement with the housing 108. The housing 108 and the diaphragm 110 cooperate to define a first variable volume chamber 112 and a second variable volume chamber 114 isolated from the first variable volume chamber 112. As shown, a portion of the housing 108 defining the first variable volume chamber 112 may be a bellows 112A. In embodiments, a portion of the housing 108 defining the second variable volume chamber 114 may be a bellows, as well (not shown).

The housing 108 defines a bi-directional port 118 fluidly connected to the first bi-directional port 16 of the oscillator 10. The bi-directional port 118 of the accumulator 104 enables communication of fluid from the oscillator 10 into the first variable volume chamber 112, and from the first variable volume chamber 112 to the oscillator 10, for example, in response to the oscillation of the oscillator 10, as discussed above. The housing 108 also defines an inlet port 120 fluidly connected to a source of breathing air BA, for example without limitation, the atmosphere. In embodiments, the breathing air may be modified with medicaments or otherwise. The inlet port 120 enables breathing air to be selectively drawn from the source of breathing air BA into the second variable volume chamber 114. An inlet check valve 124 is provided between the inlet port 120 and the source of breathing air and configured to enable flow of breathing air from the source of breathing air BA into the second variable volume chamber 114, and to check flow from the second variable volume chamber 114 to the source of breathing air BA. As shown, the inlet check valve 124 may include a biasing member 124A tending to bias the inlet check valve 124 to a flow-checking position.

The housing 108 further defines an outlet port 122 through which breathing air may be selectively expelled from the second variable volume chamber 114 to the directional control valve 106, as will be discussed further below. An outlet check valve 126 may be provided between the outlet port 122 and the directional control valve 106 to enable flow of breathing air from the second variable volume chamber 114 to the directional control valve 106 in a flow-enabling state, and to check flow from the directional control valve 106 to the second variable volume chamber 114 in a flow-checking state. As shown, the outlet check valve 126 may include a biasing member 126A tending to bias the outlet check valve 126 to the flow-checking state.

As will be discussed further below, the diaphragm 110 is displaceable between a first position corresponding to the first operational state of the accumulator 104, and a second position corresponding to the second operational state of the accumulator 104, for example, as shown in FIGS. 21A and 21B, respectively. In the first operational state, the first variable volume chamber 112 has a relatively low volume and the second variable volume chamber 114 has a relatively high volume. In the second operational state, the first variable volume chamber 112 has a relatively high volume and the second variable volume chamber 114 has a relatively low volume. The foregoing displacement of the diaphragm 110 may be referred to herein as the displacement or stroke of the diaphragm 110 (or the diaphragm displacement or stroke). The difference between the relatively high volume of the second variable volume chamber 114 in the first operational state and the relatively low volume of the second variable volume chamber 114 in the second operational state may be referred to herein as the displaceable volume of the second variable volume chamber 114.

The second variable volume chamber 114 may be configured so that the displaceable volume thereof generally corresponds to a typical user's lung capacity. In embodiments, such a typical user's lung capacity may be the lung capacity of a typical human adult. In embodiments, such a typical user's lung capacity may be the lung capacity of a typical human child. The displaceable volume of the second variable volume chamber 114 may be selected as a function of the physical size of the portion of the housing 108 defining the second variable volume chamber 114 (for example, the cross-sectional area of the portion of the housing 108 defining the second variable volume chamber 114) and the displacement or stroke of the diaphragm 110. In embodiments, the displaceable volume of the second variable volume chamber 114 may be fixed. In other embodiments, the displaceable volume of the second variable volume chamber 114 may be varied, for example, by setting the stroke of the diaphragm 110 to ones of various limits.

A biasing member 116 is operably associated with the housing 108 and the diaphragm 110. The biasing member 116 is configured to bias the diaphragm 110 to or toward the first position corresponding to the first operational state of the accumulator 104. The biasing member 116 may take any suitable form. As shown, the biasing member 116 is a coil compression spring.

The accumulator 104 is configured to adopt one of the first operational state and the second operational state in response to fluid pressure at the bi-directional port 118 thereof. More specifically, the accumulator 104 is configured to adopt the first operational state shown in FIG. 21A when the first variable volume chamber 112 is fluidly connected to the exhaust region E via the oscillator 10, as discussed above and as will be discussed further below. The accumulator 104 is configured to adopt the second operational state shown in FIG. 21B when the first variable volume chamber 112 is fluidly connected to the source of pressurized fluid FS via the control valve 10, as discussed above and as will be discussed further below.

The directional control valve 106 includes a fluid inlet port 106A, a bi-directional fluid port 106B, and a fluid outlet port 106C. The fluid inlet port 106A of the directional control valve 106 is fluidly connected to the outlet port 122 of the accumulator 104. The bi-directional fluid port 106B of the directional control valve 106 is fluidly connected to the respiratory interface 102. The fluid outlet port 106C of the directional control valve 106 is fluidly connected to an exhaust region E′ external to the directional control valve 106. The exhaust region E′ may be an environment surrounding the mechanical ventilator 100. In embodiments, the exhaust region E′ may be a return air collection system or filtering apparatus (not shown) integral with or otherwise operably associated with the mechanical ventilator 100, as would be understood by one skilled in the art. The exhaust region E′ may be, but need not be, the same exhaust region as the exhaust region E.

The optional directional control valve 106′ includes a fluid inlet port 106A′, a bi-directional fluid port 106B′, and a fluid outlet port 106C′. The fluid inlet port 106A′ of the optional directional control valve 106′ is fluidly connected to the first bi-directional port 16 of the control valve 10. The bi-directional fluid port 106B′ of the optional directional control valve 106′ is fluidly connected to the bi-directional port 118 of the accumulator 104. The fluid outlet port 106C′ of the optional directional control valve 106′ is fluidly connected to an exhaust region E′ external to the directional control valve 106′. The exhaust region E′ may be an environment surrounding the mechanical ventilator 100. In embodiments, the exhaust region E′ may be a return air collection system or filtering apparatus (not shown) integral with or otherwise operably associated with the mechanical ventilator 100, as would be understood by one skilled in the art. The exhaust region E′ may be, but need not be, the same exhaust region as the exhaust region E.

FIGS. 22A and 22B show schematically the directional control valve 106 in first and second operational states, respectively. In the first operational state shown in FIG. 22A, the directional control valve 106 is configured to enable flow therethrough from the fluid inlet port 106A thereof to the bi-directional port 106B thereof, and to thereby enable flow from the outlet port 122 of the accumulator 104 to the respiratory interface 102. Also in the first operational state, the directional control valve 106 is configured to disable flow therethrough from the fluid inlet port 106A thereof and the bi-directional port 106B thereof to the fluid outlet port 106C thereof, and to thereby disable flow from the outlet port 122 of the accumulator 104 and the respiratory interface 102 to the exhaust region E′ external to the directional control valve 106 (and vice versa). This first operational state corresponds to an inhale portion of the predetermined breathing cycle.

In the second operational state shown in FIG. 22B, the directional control valve 106 is configured to enable flow therethrough from the bi-directional port 106B thereof to the fluid outlet port 106C thereof, and to thereby enable flow from the first respiratory interface 102 to the exhaust region E′ external to the directional control valve 106. Also in the second operational state, the directional flow control valve 106 is configured to disable flow therethrough from the bi-directional port 106B thereof and the fluid outlet port 106C thereof to the fluid inlet port 106A thereof, and to thereby disable flow from the first respiratory interface 102 and the exhaust region E′ to the outlet port 122 of the accumulator 104 (and vice versa). This second operational state corresponds to an exhale portion of the predetermined breathing cycle.

The directional control valve 106 is configured to adopt one of the first operational state and the second operational state in response to fluid pressure at the fluid inlet port 106A thereof. More specifically, when the fluid pressure at the fluid inlet port 106A of the directional control valve 106 is above a transition pressure, as will be discussed further below, the directional control valve 106 adopts the first operational state. Conversely, when the fluid pressure at the fluid inlet port 106A of the directional control valve 106 is below the transition pressure, as will be discussed further below, the directional control valve 106 adopts the second operational state.

Prior to use, the accumulator 104 is in the first operational state, for example, as shown in FIG. 21A, the directional control valve 106 is in the second operational state, for example, as shown in FIG. 22B, and each of the inlet check valve 124 and the optional outlet check valve 126 is in the flow-disabling state.

The optional directional control valve 106′ may be functionally and operationally identical to the directional control valve 106.

In use, the oscillator 10 cycles between first and second operational states, as described further above. With the oscillator 10 initially in the first operational state, the oscillator 10 enables flow of pressurized fluid from the source of pressurized fluid FS from the inlet port 14 thereof to the first bi-directional port 16 thereof, and it disables flow to the outlet port 20 thereof and the exhaust region E external to the oscillator 10, as discussed further above. Consequently, the pressurized fluid may flow from the source of pressurized fluid FS, through the oscillator 10, through the bi-directional port 118 of the accumulator 104, and into the first variable volume chamber 112 of the accumulator 104.

This flow of pressurized fluid entering the first variable volume chamber 112 causes the fluid pressure within the first variable volume chamber 112 to increase. This increasing pressure causes the diaphragm 110 to be displaced in the direction of the second variable volume chamber 114, thereby increasing the volume of the first variable chamber 112 and decreasing the volume of the second variable volume chamber 114. This displacement of the diaphragm 110 causes the pressure of breathing air in the second variable volume chamber 114 to increase. This increase in breathing air pressure in the second variable volume 114 causes the inlet check valve 124 to assume or maintain the flow-checking state, and it causes the outlet check valve 126 to assume or maintain the flow-enabling state. Consequently, the pressure of the pressurized breathing air at the fluid inlet port 106A of the directional control valve 106 rises above the transition pressure, causing the directional control valve 106 to adopt the first operational state, as discussed above, thereby enabling flow of breathing air from the second variable volume chamber 114 of the accumulator 104, through the outlet check valve 126, through the directional control valve 106, to the respiratory interface 102, and to a user thereof.

The oscillator 10 subsequently adopts the second operational state, as discussed further above. With the oscillator 10 in the second operational state, the oscillator 10 disables flow from the source of pressurize fluid FS to the first bi-directional port thereof, and it enables flow of fluid from the first bi-directional port 16 thereof to the exhaust port 20 thereof and exhaust region E external to the oscillator 10.

With the oscillator 10 in the second operational state, the biasing member 116 causes the diaphragm 110 to be displaced in the direction of the first variable volume chamber 112, thereby decreasing the volume of the first variable chamber 112 and increasing the volume of the second variable volume chamber 114. This displacement of the diaphragm 110 causes the pressure of the fluid in the first variable volume chamber 112 to increase, thereby forcing fluid out of the first variable volume chamber 112, through the bi-directional port 118 of the accumulator, and through the oscillator 10 from the first bi-directional port 16 to the exhaust port 20 and the exhaust region E. In embodiments including the optional directional control valve 106′, which may be functionally identical to the directional control valve 106, the fluid forced out of the first variable volume chamber 112 may instead be discharged directly to the exhaust region E′. This displacement of the diaphragm 110 also causes the pressure of breathing air in the second variable volume chamber 114 to decrease. This decrease in breathing air pressure in the second variable volume 114 causes the inlet check valve 124 to assume or maintain the flow-enabling state, and it causes the outlet check valve 126 to assume or maintain the flow-disabling state. Consequently, breathing air is drawn from the source of breathing air BA, through the inlet check valve 124, and into the second variable volume chamber 114. Also, the pressure at the fluid inlet port 106A of the directional control valve 106 decreases below the transition pressure, thereby causing the directional control valve 120 to adopt or maintain the second operational state, as discussed above. (In embodiments, a pressure-bleeding orifice (not shown) may be provided in fluid communication with the fluid line connecting the outlet check valve 126 to the inlet port 106A of the directional control valve 106 to assist in lowering the pressure at the fluid inlet port 106A below the transition pressure by venting breathing air in the fluid line out of the fluid line, for example, to the environment surrounding the fluid line or to the second variable volume chamber 114.) With the directional control valve 120 in the second operational state, flow is enabled from the respiratory interface 102 and the user thereof, through the directional control valve 106 via the bi-directional fluid port 106B and the fluid outlet port 106C thereof to the exhaust region E′.

The oscillator 10, the accumulator 104, and the directional control valve 106 subsequently re-adopt their respective first and second operational states, as discussed above, so long as the oscillator 10 is provided with pressurized fluid, as discussed above.

In embodiments, the oscillator 10 could be eliminated and the bi-directional port 118 of the accumulator 104 could instead be connected to another source of pressurized fluid configured to be selectively admitted to and relieved from the first variable volume chamber 112 of the accumulator 104 to thereby effect selective displacement of the diaphragm 110 in a manner similar to that described above.

In embodiments including a second respiratory interface 102 and related components, as discussed further above, the first respiratory interface 102 may be configured for use by a second user.

The disclosure illustrates and describes an oscillating flow control valve internally configured to receive pressurized fluid through a fluid input port and alternatingly provide pressurized fluid output to an accumulator through a first bi-directional port. The oscillating flow control valve is passive, that is, it requires no external power other than the pressurized fluid to effect the oscillating output thereof. The benefits of the disclosure may be realized using flow control valves having other internal configurations that enable an oscillating or cyclic output similar to that described herein. The benefits of the disclosure also may be realized using other flow controllers or flow control mechanisms valves that enable an oscillating or cyclic output similar to that described herein.

The embodiments shown and described herein are illustrative and not limiting. One skilled in the art would recognize that features of any embodiment may be freely interchanged with features of other embodiments without departure from the scope of the appended claims. One skilled in the art also would recognize that any embodiment may be readily modified without departure from the scope of the appended claims.

1. A mechanical ventilator comprising;
an oscillating flow controller comprising a supply port, a first bi-directional port, and an exhaust port, wherein the oscillating flow controller is configured to receive pressurized fluid through the supply port, selectively output the pressurized fluid through the first bi-directional port, selectively receive the pressurized fluid through the first-bi-directional port, and selectively output the pressurized fluid though the exhaust port;
an accumulator comprising:
a housing;
a first variable volume chamber defined within the housing;
a second variable volume chamber defined within the housing;
a diaphragm separating the first variable volume chamber from the second variable volume chamber;
a biasing element configured to bias the diaphragm toward the first variable volume chamber;
a bi-directional fluid port configured to admit fluid to the first variable volume chamber from the first bi-directional port of the control valve, and to relieve fluid from the first variable volume chamber to the first bi-directional port of the control valve;
a fluid inlet port configured to admit breathing air to the second variable volume chamber;
a fluid outlet port configured to relieve breathing air from the second variable volume chamber; and
a first respiratory interface having a first breathing air conduit, a first end of the first breathing air conduit fluidly coupled to the fluid outlet port and a second end of the first breathing air conduit configured to supply the breathing air from the fluid outlet port to a respiratory system of a first user,
wherein the oscillating flow controller is configured to alternate between a first state in which the oscillating flow controller enables flow of the pressurized fluid from the supply port through the first bi-directional port to the first variable volume chamber and disables flow of the pressurized breathing air from the supply port and the first bi-directional port to the exhaust port, and a second state in which the oscillating flow controller enables flow of the pressurized fluid from the first variable volume chamber through the first bi-directional port to the exhaust port and disables flow of the pressurized breathing air from the supply port to the first bi-directional port and the exhaust port, and
wherein the oscillating flow controller further is configured to cyclically change state between the first state and the second state in response to the pressurized fluid flowing therethrough.

2. The mechanical ventilator of claim 1 wherein the oscillating flow controller is configured to oscillate according to a predetermined breathing rhythm.

3. The mechanical ventilator of claim 1 further comprising a first check valve fluidly connected to the fluid inlet port and configured to enable flow of breathing air from a source of breathing air to the second variable volume chamber through the fluid inlet port and to disable fluid flow from the second chamber through the fluid inlet port.

4. The mechanical ventilator of claim 3 further comprising a second check valve configured to enable flow of the breathing air from the second variable volume chamber to the first respiratory interface through the fluid outlet port and to disable flow from the first respiratory interface to the second variable volume chamber through the fluid inlet port.

5. The mechanical ventilator of claim 1 further comprising a directional flow control valve fluidly connected between the fluid outlet port and the respiratory interface, the directional flow control valve comprising a fluid inlet port, a bi-directional port, and a fluid outlet port.

6. The mechanical ventilator of claim 5, wherein the directional flow control valve is configured to selectively adopt (i) a first state wherein the directional flow control valve enables flow from the fluid inlet port thereof to the bi-directional port thereof while disabling flow from the bi-directional port thereof to the fluid outlet port thereof, and (ii) a second state wherein the directional flow control valve enables flow from the bi-directional port thereof to the fluid outlet port thereof while disabling flow from the fluid inlet port thereof to the bi-directional port thereof.

7. The mechanical ventilator of claim 5, wherein the directional flow control valve is configured to selectively adopt one of the first state and the second in response to fluid pressure at the fluid inlet port thereof.

8. The mechanical ventilator of claim 6 further comprising a second directional control valve, the second directional control valve fluidly connected between the first bi-directional port and the bi-directional fluid port, the second directional flow control valve comprising a fluid inlet port, a bi-directional port, and a fluid outlet port.

9. A mechanical ventilator comprising:
a fluid source configured to selectively supply pressurized fluid;
an accumulator, the accumulator comprising:
a housing;
a first variable volume chamber defined within the housing;
a second variable volume chamber defined within the housing
a diaphragm separating the first variable volume chamber from the second variable volume chamber;
a biasing element configured to bias the diaphragm toward the first variable volume chamber;
a bi-directional fluid port configured to admit pressurized fluid to the first variable volume chamber from the fluid source, and to relieve fluid from the first variable volume chamber;
a fluid inlet port configured to admit breathing air to the second variable volume chamber;
a fluid outlet port configured to relieve breathing air from the second variable volume chamber; and
a respiratory interface fluidly connected to the fluid outlet port.

10. The mechanical ventilator of claim 9, wherein the fluid source is configured to selectively supply the pressurized fluid according to a predetermined breathing rhythm.

11. The mechanical ventilator of claim 9 further comprising a first check valve fluidly connected to the fluid inlet port and configured to enable flow of breathing air from a source of breathing air to the second variable volume chamber through the fluid inlet port and to disable fluid flow from the second chamber through the fluid inlet port.

12. The mechanical ventilator of claim 11 further comprising a second check valve configured to enable flow of the breathing air from the second variable volume chamber to the first respiratory interface through the fluid outlet port and to disable flow from the first respiratory interface to the second variable volume chamber through the fluid inlet port.

13. The mechanical ventilator of claim 9, wherein the fluid source is an oscillating flow controller.

14. The mechanical ventilator of claim 13 wherein the oscillating flow controller is configured to oscillate according to a predetermined breathing rhythm.

15. The mechanical ventilator of claim 13, wherein the oscillating flow controller comprises a supply port, a first bi-directional port, and an exhaust port, and wherein the oscillating flow controller is configured to receive pressurized fluid through the supply port, selectively output the pressurized fluid through the first bi-directional port, selectively receive the pressurized fluid through the first-bi-directional port, and selectively output the pressurized fluid though the exhaust port.

16. The mechanical ventilator of claim 9 further comprising a directional control valve fluidly coupled between the fluid source and the bi-directional port, the directional flow control valve comprising a fluid inlet port fluidly coupled to the fluid source, a bi-directional port fluidly coupled to the respiratory interface, and a fluid outlet port fluidly coupled to an environment external to the mechanical ventilator.

17. The mechanical ventilator of claim 16, wherein the directional flow control valve is configured to selectively adopt (i) a first state wherein the directional flow control valve enables flow from the fluid inlet port thereof to the bi-directional port thereof while disabling flow from the bi-directional port thereof to the fluid outlet port thereof, and (ii) a second state wherein the directional flow control valve enables flow from the bi-directional port thereof to the fluid outlet port thereof while disabling flow from the fluid inlet port thereof to the bi-directional port thereof.
